(19) United States
(12) Patent Application Publication
(10) Pub. No.: US 2024/0091126 A1
(43) Pub. Date: Mar. 21, 2024

(54) RESIN COMPOSITION FOR COSMETIC, RESIN COMPOSITION FOR NAIL, BASE COAT AGENT FOR GEL NAIL, AND ADHESIVE FOR EYELASH EXTENSION

(71) Applicant: Panasonic Intellectual Property Management Co., Ltd., Osaka (JP)

(72) Inventors: Tomoko KAWASHIMA, Osaka (JP); Taiki UMEMOTO, Shiga (JP); Keitaro AMARI, Kyoto (JP); Yuko TANIIKE, Osaka (JP); Haruka KUSUKAME, Nara (JP); Shinsuke KAWAGUCHI, Shiga (JP); Toshiyuki KOMATSU, Kyoto (JP)

(21) Appl. No.: 18/263,074

(22) PCT Filed: Jan. 31, 2022
(86) PCT No.: PCT/JP2022/003571

§ 371 Date: Jul. 26, 2023

(30) Foreign Application Priority Data
Feb. 4, 2021 (JP) 2021-016945
Aug. 25, 2021 (JP) 2021-137356

(51) Int. Cl. A61K 8/86; A61Q 1/10; A61Q 3/02

(52) U.S. Cl. CPC A61K 8/86; A61Q 1/10; A61Q 3/02; A61K 2800/22

(57) ABSTRACT

Base coat as an example of a resin composition for a cosmetic is a resin composition for a cosmetic used in a cosmetic to be adhered to nail as a part of living body. The resin composition for a cosmetic includes a stimulus-responsive material that undergoes a volume change by external stimuli, the external stimuli excluding both an external stimulus due to contact with a solvent and an external stimulus due to contact with a solution, and a solidified matter of the resin composition for a cosmetic has a surface with a water contact angle between 30° and 110° (inclusive). The solidified matter of the resin composition for a cosmetic undergoes the volume change by application of the external stimuli to reduce strength of adhesion to the part of living body.

### TECHNICAL FIELD

The present disclosure relates to a resin composition for a cosmetic, a resin composition for a nail, a base coat agent for a gel nail, and an adhesive for an eyelash extension.

### BACKGROUND ART

In recent years, many people use cosmetics that can be continuously worn on a living body for a period of several days to several weeks, such as gel nails and eyelash extensions. In a conventional technique, for example, a cosmetic for a nail such as the gel nail firmly adheres to an own nail by using a photocurable resin or the like. Furthermore, a lash cosmetic such as the eyelash extension firmly adheres to eyelashes by using a humidity curable resin or the like. When these cosmetics are peeled off from the living body, it is common to use a solvent such as acetone. When the cosmetic for a nail is peeled off from the living body, polishing is often performed before or after the solvent is used.

PTL 1 proposes an artificial nail composition that allows an artificial nail to firmly adhere to the own nail by using radical polymerization.

PTL 2 discloses an artificial nail raw material composition that includes a radical polymerizable compound, a polyfunctional thiol compound having a thiol group, and a photopolymerization initiator. By using the artificial nail raw material composition, an adhesive property of an artificial nail to the own nail can be maintained for a long period without using an adhesive and a primer. When the artificial nail obtained by curing the artificial nail raw material composition is removed, a method is used to firstly scrap and scratch the artificial nail, then to dispose cotton immersed in acetone on the artificial nail for about 10 minutes, and then to peel off the artificial nail from the own nail by using, for example, a stick.

PTL 3 discloses an artificial nail composition that includes an ionic monomer polymerizable by irradiation with ultraviolet rays. An artificial nail obtained by curing the artificial nail composition can be removed from the own nail with an acid having a pH of less than or equal to 3.5.

PTL 4 proposes a method and a kit for treating a nail surface, capable of removing a cover (for example, a gel nail or an artificial nail) applied to the nail surface by solvent immersion for less than 20 minutes, peeling, or a combination thereof.

As an adhesive for an eyelash extension, for example, cyanoacrylate is generally used. The cyanoacrylate is an adhesive having high adhesive force, and is cured to adhere by polymerization in accordance with humidity, for example, in air. The cyanoacrylate is called an instant adhesive and widely used not only in industrial and medical applications but also in general households. As for the eyelash extension or the like, artificial lashes can adhere to the eyelashes for a period of several weeks by allowing the lashes to firmly adhere to the artificial lashes by using a cyanoacrylate adhesive. PTL 5 discloses a cyanoacrylate-containing composition that includes cyanoacrylate and fullerene. The cyanoacrylate-containing composition can be used as an adhesive composition used for eyelash extensions or the like, and is also excellent in storage stability.

### CITATION LIST

### Patent Literature

PTL 1: Unexamined Japanese Patent Publication No. 2010-53097
PTL 2: Unexamined Japanese Patent Publication No. 2014-5260
PTL 3: Unexamined Japanese Patent Publication No. 2009-5260
PTL 4: Japanese Translation of PCT International Application No. 2019-512000
PTL 5: Unexamined Japanese Patent Publication No. 2016-121299

### SUMMARY OF THE INVENTION

Resin compositions for cosmetics, such as a photocurable resin used in gel nails and cyanoacrylate as a humidity curable resin used in eyelash extensions can adhere to a living body such as nails and lashes with high strength. However, in order to peel off the resin compositions for cosmetics from the living body, it is necessary to perform polishing or to use a solvent such as acetone, an acid, or the like, and it has been a large burden for the living body such as the nails and the lashes to be subjected to peeling treatment. On the other hand, in a case where the adhesive property of the resin composition for a cosmetic to the living body is reduced in order to facilitate the peeling treatment, there arises a problem that the cosmetic cannot be continuously worn on the living body for a desired period.

The present disclosure provides a resin composition for a cosmetic that can firmly adhere to a part of living body and can be easily peeled off from the living body at any timing.

The present disclosure is a resin composition for a cosmetic in a cosmetic to adhere to the part of living body, and the resin composition for a cosmetic includes a stimulus-responsive material undergoing a volume change by external stimuli, the external stimuli excluding both an external stimulus due to contact with a solvent and an external stimulus due to contact with a solution. Provided is a resin composition for a cosmetic, a solidified matter of the resin composition for a cosmetic having a surface with a water contact angle between 30° and 110° (inclusive), and the solidified matter of the resin composition for a cosmetic undergoing the volume change by application of the external stimuli to reduce strength of adhesion of the part of living body to the solidified matter.

In another aspect, the present disclosure provides a resin composition for a nail, including the resin composition for a cosmetic of the present disclosure described above.

In another aspect, the present disclosure provides a base coat agent for a gel nail, including the resin composition for a cosmetic of the present disclosure described above.

In another aspect, the present disclosure provides an adhesive for an eyelash extension, including the resin composition for a cosmetic of the present disclosure described above.

The resin composition for a cosmetic in the present disclosure can firmly adhere to the part of living body, and can be easily peeled off from the living body at any timing.

### BRIEF DESCRIPTION OF THE DRAWINGS

FIG. 1 is a schematic sectional view illustrating a state where a base coat and a gel nail adhere to a nail when a resin composition for a cosmetic in an exemplary embodiment is used as a base coat agent for a gel nail.

FIG. 2 is a schematic sectional view illustrating a state where a base coat and a gel nail including a color gel nail and a top gel nail adhere to a nail when a resin composition for a cosmetic in an exemplary embodiment is used as a base coat agent for a gel nail.

FIG. 3 is a schematic sectional view illustrating a state where a base coat and a gel nail adhere to a nail via a thin membrane layer when a resin composition for a cosmetic in an exemplary embodiment is used as a base coat agent for a gel nail.

FIG. 4 is a schematic sectional view explaining an example of a principle of a decrease in strength of adhesion of a base coat to a nail when a resin composition for a cosmetic in an exemplary embodiment is used as a base coat agent for a gel nail.

FIG. 5 is a graph illustrating a relationship between an strength of adhesion ratio after irradiation with ultraviolet light in a case where the strength of adhesion before the irradiation with the ultraviolet light is 100% and an area ratio of the stimulus-responsive material to an adhesion area, for a resin composition for a cosmetic in Examples 1 to 6 and an acrylic polymer in Comparative Example 1.

### DESCRIPTION OF EMBODIMENT

Hereinafter, an exemplary embodiment will be described in detail with reference to drawings. However, unnecessary detailed descriptions may be omitted. For example, detailed descriptions of already well-known matters or redundant descriptions of substantially the same configuration may be omitted. This is to avoid an unnecessary redundancy in the following description and to facilitate understanding of those skilled in the art.

Note that, the accompanying drawings and the following description are merely presented to help those skilled in the art fully understand the present disclosure, and are not intended to limit the subject matters described in the scope of claims.

### Exemplary Embodiment

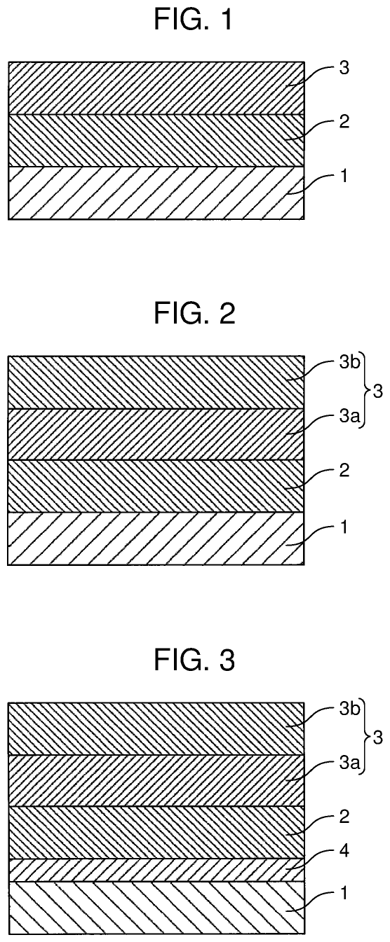

An exemplary embodiment will be described below with reference to FIGS. 1 to 3.

[1-1. Configuration]

A resin composition for a cosmetic in the exemplary embodiment is used in a cosmetic to be adhered to a part of living body. The resin composition for a cosmetic in the present exemplary embodiment includes a stimulus-responsive material undergoing a volume change by external stimuli. A solidified matter of the resin composition for a cosmetic in the present exemplary embodiment has a surface with a water contact angle between 30° and 110° (inclusive). The solidified matter of the resin composition for a cosmetic in the present exemplary embodiment undergoes the volume change by application of the external stimuli described above to reduce strength of adhesion to the part of living body. Note that the external stimuli here are external stimuli, the external stimuli excluding both an external stimulus due to contact with a solvent and an external stimulus due to contact with a solution. Furthermore, a plurality of stimuli may be combined as the external stimulus. With such configuration, the resin composition for a cosmetic in the present exemplary embodiment can firmly adhere to the part of living body, and can be easily peeled off from the living body at any timing.

As described above, the resin composition for a cosmetic in the present exemplary embodiment has the water contact angle between 30° and 110° (inclusive) in a solidified state. The contact angle can be measured by using a contact angle meter. The water contact angle is measured by a droplet method. Specifically, an angle between the surface of the solidified matter and the water is obtained by measured with the contact angle meter, for example, in a case where 5 μL of the water is added dropwise to a flat surface of the solidified matter of the resin composition for a cosmetic. The water contact angle can generally represent a permutation of a hydrophilic property and a hydrophobic property of a material, and also has some correlation with physical properties of the material, such as solubility parameters (SP values). It is generally known that a material having the hydrophilic property close to the hydrophobic property and a material having the SP values close to each other tend to have good compatibility between the materials as adhesive force increases. A material having the water contact angle between 30° and 110° (inclusive) has a contact angle property close to a contact angle property of the living body such as the nail and the lash. Therefore, the resin composition for a cosmetic in the present exemplary embodiment has high adhesive force to the living body in the solidified state, and can stably adhere to the living body for a long time. In a case where the solidified matter of the resin composition for a cosmetic in the present exemplary embodiment has a water contact angle between 40° and 110° (inclusive), the resin composition for a cosmetic in the present exemplary embodiment can more firmly adhere to the living body. In a case where the solidified matter of the resin composition for a cosmetic in the present exemplary embodiment has a water contact angle between 60° and 100° (inclusive), the resin composition for a cosmetic in the present exemplary embodiment can more firmly adhere to the living body.

Furthermore, the resin composition for a cosmetic in the present exemplary embodiment includes the stimulus-responsive material undergoing the volume change by the external stimuli, as described above. Since such stimulus-responsive material is included, the solidified matter of the resin composition for a cosmetic in the present exemplary embodiment undergoes the volume change by the application of the external stimuli to reduce the strength of adhesion of the part of living body to the resin composition for a cosmetic. With such configuration, the resin composition for a cosmetic in the present exemplary embodiment can be easily peeled off from the living body by applying the external stimuli at any timing when the composition is desired to be peeled off from the living body. The stimulus-responsive material may be a material that causes at least one selected from the group consisting of a change in physical property and a chemical reaction by the external stimuli. Examples of the change in physical property include vaporization, sublimation, a change in density, liquefaction, and a change in viscoelasticity. Examples of the chemical reaction include a reaction accompanied with a change in molecular structure and a decomposition reaction. The stimulus-responsive material may be a material that generates a gas due to, for example, the chemical reaction or sublimation caused by the external stimuli. The gas is generated to cause the volume change.

The solidified matter of the resin composition for a cosmetic in the present exemplary embodiment means, for example, a solid such as a coating membrane obtained by volatilizing a solvent of the resin composition for a cosmetic in a case where the resin composition for a cosmetic includes the solvent and is a resin composition capable of forming a solid such as the coating membrane by volatilizing the solvent. Furthermore, in a case where the resin composition for a cosmetic in the present exemplary embodiment is, for example, a curable resin composition that includes a polymerizable compound and can be cured by light or heat, the solidified matter of the resin composition for a cosmetic in the present exemplary embodiment means a cured product of the resin composition for a cosmetic cured by being irradiated with light or heat.

In the resin composition for a cosmetic in the present exemplary embodiment, the cosmetic to adhere to the part of living body may be a cosmetic that directly adheres to the part of living body, or may be a cosmetic that indirectly adheres to the living body via another layer such as a thin membrane between the cosmetic and the living body. For example, in a case where the cosmetic is a cosmetic for a nail, the thin membrane as the another layer may be, for example, at least one selected from the group consisting of a primer, a base gel, and a base coat.

The external stimulus is, for example, at least one selected from the group consisting of light, heat, magnetic force, electricity, and an external force. In accordance with these external stimuli, the peeling can be easier, and direct damage to the living body is less likely to occur. As described above, a plurality of stimuli may be combined as the external stimulus. Therefore, for example, the light and the heat may be simultaneously used as the external stimuli, or a plurality of types of light having different wavelengths may be used. In a case where the light is used as the external stimulus, for example, ultraviolet light, visible light, near infrared light, infrared light, or far infrared light can be used. In a case where the heat is used as the external stimulus, for example, a heat source, hot air, near infrared light, infrared light, far infrared light, or microwave can be used as a way for applying the heat. The external force is, for example, an impact.

A degree of the external stimulus to be applied is not particularly limited. The degree of the external stimulus to be applied may be appropriately adjusted in accordance with a type of the stimulus-responsive material included in the resin composition for a cosmetic in the present exemplary embodiment and a content ratio of the stimulus-responsive material. Furthermore, the degree of the external stimulus to be applied may be changed in accordance with the water contact angle with respect to the surface of the solidified matter of the resin composition for a cosmetic in the present exemplary embodiment.

The external stimulus may be at least one selected from the group consisting of light and heat. The light and the heat can be easily applied with simple equipment. Furthermore, high safety for the living body can be ensured by appropriately selecting a wavelength of the light. For example, the light having a wavelength of more than or equal to 280 nm has the high safety for the living body. Furthermore, the high safety for the living body can be ensured by appropriately selecting a temperature of the heat. A temperature range of the heat capable of being applied as the external stimulus may be appropriately determined in accordance with the part of living body with the cosmetic adhered. For example, in a case where the cosmetic adheres to lashes, for example, the safety for the living body is high at a temperature of less than or equal to 250° C. Furthermore, at a temperature of less than or equal to 100° C., since the safety for not only the lashes but also the living body such as nails and skins is higher, many resin compositions for cosmetics to adhere to the living body can be safely utilized as the external stimuli. Therefore, in a case where the external stimulus is the light or the heat, treatment of peeling off the solidified matter of the resin composition for a cosmetic from the living body becomes easier, and the direct damage to the living body can be further suppressed.

As the external stimuli, for example, light having a peak wavelength ranging from 280 nm up to, not including 400 nm may be used. As described above, the light can be easily applied with simple equipment. Furthermore, light having a wavelength of more than or equal to 280 nm has the high safety for the living body. Furthermore, a fact that light having a wavelength of less than 400 nm can be used as the external stimulus can be regarded as that the stimulus-responsive material included in the resin composition for a cosmetic in the present exemplary embodiment is a substance undergoing the volume change with light having the wavelength of less than 400 nm, and is a substance difficultly undergoing the volume change to an extent that the strength of adhesion to the living body is reduced with light having a wavelength of more than or equal to 400 nm. Therefore, in this case, it is possible to prevent a decrease in strength of adhesion in a normal living environment such as general lighting while implementing application of a stimulus having high energy with a wavelength of about 400 nm. Accordingly, in a case where the light having the peak wavelength ranging from 280 nm up to, not including 400 nm is used as the external stimulus, it is possible to implement effective peeling at any timing by a simpler method and to further suppress the damage to the living body while preventing a decrease in strength of adhesion of the resin composition for a cosmetic to the living body in the normal living environment.

In a case where the light having the peak wavelength ranging from 280 nm up to, not including 400 nm is used as the external stimulus, the solidified matter of the resin composition for a cosmetic in the present exemplary embodiment may have a transmittance of, for example, more than or equal to 10% for the light having any wavelength ranging from 280 nm up to, not including 400 nm. Since the solidified matter of the resin composition for a cosmetic in the present exemplary embodiment has such transmittance, the external stimuli effectivity cause the volume change of the stimulus-responsive material, and as a result, the strength of adhesion of the solidified matter of the resin composition for a cosmetic to the part of living body is effectivity reduced. In other words, according to the configuration, the resin composition for a cosmetic in the present exemplary embodiment can be effectively peeled off from the living body. The solidified matter of the resin composition for a cosmetic in the present exemplary embodiment may have a transmittance of more than or equal to 30%, or may have a transmittance of more than or equal to 50% for the light having any wavelength ranging from 280 nm up to, not including 400 nm. In a case where the transmittance is more than or equal to 30%, since the external stimuli cause the volume change of the stimulus-responsive material more effectively, the strength of adhesion of the solidified matter of the resin composition for a cosmetic to the part of living body is reduced more effectively. In a case where the transmittance is more than or equal to 50%, since the external stimuli cause the volume change of the stimulus-responsive material still more effectively, the strength of adhesion of the solidified matter of the resin composition for a cosmetic to the part of living body is reduced still more effectively. Furthermore, since the solidified matter of the resin composition for a cosmetic in the present exemplary embodiment has a high transmittance, an effect of increasing transparency and improving designability is also obtained.

The light used as the external stimulus may be, for example, light having a peak wavelength between 280 nm and 365 nm (inclusive). Since the wavelength of the light applied as the external stimulus is less than or equal to 365 nm, it is possible to more reliably prevent a decrease in strength of adhesion in the normal living environment such as the general lighting while implementing application of a stimulus having high energy with a wavelength of about 365 nm. In other words, in a case where the light having the peak wavelength between 280 nm and 365 nm (inclusive) is used as the external stimulus, it is possible to implement the effective peeling at any timing by a simpler method and to further suppress the damage to the living body while more reliably preventing a decrease in strength of adhesion of the resin composition for a cosmetic to the living body in the normal living environment. In addition, since light having a peak wavelength of less than or equal to 365 nm is used as the external stimulus, for example, in a case where the resin composition for a cosmetic in the present exemplary embodiment is used as a base coat agent for a gel nail, an effect that can prevent a decrease in strength of adhesion of the base coat during gel nail curing is also obtained. In this case, the solidified matter of the resin composition for a cosmetic in the present exemplary embodiment may, for example, have the transmittance of more than or equal to 10%, or may have the transmittance of more than or equal to 30%, or may have the transmittance of more than or equal to 50% for the light having any wavelength between 280 nm and 365 nm (inclusive). Since the solidified matter of the resin composition for a cosmetic in the present exemplary embodiment has the above transmittance with respect to the wavelength of the light used as the external stimulus, the resin composition for a cosmetic in the present exemplary embodiment can be effectively peeled off from the living body by application of the external stimulus.

The light used as the external stimulus may be, for example, the light having a peak wavelength between 280 nm and 350 nm (inclusive). Since the peak wavelength of the light used as the external stimulus is less than or equal to 350 nm, irradiation with higher energy causes more volume change. Accordingly, it possible to more effectively peel off the resin composition for a cosmetic in the present exemplary embodiment from the living body.

Since the stimulus-responsive material included in the resin composition for a cosmetic in the present exemplary embodiment may be any material that undergoes the volume change by the external stimuli to be applied, the stimulus-responsive material can be appropriately selected in accordance with the external stimuli to be used. However, in consideration of contact with the living body, the stimulus-responsive material is required to have high biocompatibility.

Examples of the stimulus-responsive material undergoing the volume change by the application of the light such as ultraviolet light or visible light as the external stimulus include an azide compound such as a glycidyl azide polymer and an azo compound. The azide compound is an organic or inorganic compound having an azide group, and decomposes to generate a nitrogen gas. Furthermore, the azo compound is an organic or inorganic compound having an azo group, and decomposes to generate the nitrogen gas.

Examples of the stimulus-responsive material undergoing the volume change by the application of the heat include (I) low-boiling materials such as petroleum ether, pentane, hexane, heptane, fatty acid hydrocarbon, methylsilane, and halogen hydrocarbon, and (II) thermally decomposable foamable materials such as azo compounds, hydrazine derivatives, nitroso compounds, azide compounds, tetrazole compounds, semicarbazide compounds, carbonates, and bicarbonates. For example, the foamable material may be encapsulated with a thermoplastic resin such as (meth)acrylic acid ester, acrylonitrile, and vinylidene chloride in order to prevent volatilization of the stimulus-responsive material at room temperature and to be stably dispersed in the resin composition for a cosmetic. Furthermore, as the stimulus-responsive material undergoing the volume change, a shape memory polymer having a property that the volume is different above and below a glass transition point may be used. Examples of the shape memory polymer include a polyurethane-based shape memory polymer having a volume expanding due to heating at more than or equal to the glass transition point.

The stimulus-responsive material may undergo the volume change by using near infrared light and infrared light as the external stimuli and applying these types of light, and the stimulus-responsive material may undergo the volume change due to heat by using the near infrared light or the infrared light to increase a temperature of the resin composition for a cosmetic. For example, a material easily absorbing the near infrared light or the infrared light may be added to the resin composition for a cosmetic. Accordingly, by applying the near infrared light or the infrared light, an effect of further increasing the temperature of the resin composition for a cosmetic may be obtained.

Examples of the material absorbing the near infrared light or the infrared light to increase the temperature include a cyanine compound, a phthalocyanine compound, a naphthalocyanine-based compound, a porphyrin derivative, a naphthoquinone compound, an anthraquinone compound, a squarylium-based compound, an immonium compound, a diimmonium compound, a triallylmethane-based compound, an azo compound, a dithiol metal complex, and nanoparticles such as carbon or gold.

The content ratio of the stimulus-responsive material in the resin composition for a cosmetic in the present exemplary embodiment can be appropriately selected to enable the volume change that can effectively reduce the strength of adhesion of the solidified matter of the resin composition for a cosmetic to the living body in a case where the external stimuli are applied. For example, the resin composition for a cosmetic in the present exemplary embodiment may include more than or equal to 0.5 vol % of the stimulus-responsive material. Since the stimulus-responsive material is included in an amount of more than or equal to 0.5 vol %, for example, the stimulus-responsive material can be present in an area of more than or equal to 0.5% of an area of an adhesion portion between the solidified matter of the resin composition for a cosmetic and the living body. Accordingly, when the external stimuli are applied, the solidified matter of the resin composition for a cosmetic can undergo the volume change in more than or equal to 0.5% of the area of the adhesion portion between the solidified matter of the resin composition for a cosmetic and the living body. Therefore, when the external stimuli are applied, the strength of adhesion of the solidified matter of the resin composition for a cosmetic to the living body can be effectively reduced, and the resin composition for a cosmetic can be more easily peeled off from the living body. The resin composition for a cosmetic in the present exemplary embodiment may include more than or equal to 0.9 vol % of the stimulus-responsive material. Since the stimulus-responsive material is included in an amount of more than or equal to 0.9 vol %, when the external stimuli are applied, the solidified matter of the resin composition for a cosmetic can undergo the volume change in more than or equal to 0.9% of the area of the adhesion portion between the solidified matter of the resin composition for a cosmetic and the living body. Therefore, when the external stimuli are applied, the strength of adhesion of the solidified matter of the resin composition for a cosmetic to the living body can be reduced more effectively, and the resin composition for a cosmetic can be still more easily peeled off from the living body.

The resin composition for a cosmetic in the present exemplary embodiment may include only the stimulus-responsive material. In other words, the resin composition for a cosmetic in the present exemplary embodiment may include the stimulus-responsive material at a ratio of 100 vol %. The resin composition for a cosmetic in the present exemplary embodiment may include the stimulus-responsive material at a ratio of less than or equal to 100 vol % or less than or equal to 50 vol %.

The content ratio of the stimulus-responsive material in the resin composition for a cosmetic can be confirmed by, for example, nuclear magnetic resonance (NMR), infrared spectroscopy (IR), mass spectrometry (MS), secondary ion mass spectrometry (SIMS), inductively coupled plasma mass spectrometry (ICP-MS), scanning electron microscopy (SEM), scanning electron microscopy-energy dispersive X-ray spectrometry (SEM-EDX), liquid chromatography, or spectrophotometry. Furthermore, whether or not the stimulus-responsive material is dissolved or uniformly dispersed in the resin composition for a cosmetic can be confirmed by transmission uniformity of the light. Whether or not the stimulus-responsive material is dissolved or uniformly dispersed in the resin composition for a cosmetic can also be confirmed by using a refractive index. In a case where the stimulus-responsive material is a material having a crystal structure, an X-ray diffraction method (XRD) can also be used. In addition, it is also possible to evaluate solubility by using permeability or viscosity of a filter.

The solidified matter of the resin composition for a cosmetic in the present exemplary embodiment may undergo the volume change in more than or equal to 0.5% of the area of the adhesion portion with the part of living body by the application of the external stimuli. Since the solidified matter of the resin composition for a cosmetic undergoes the volume change in more than or equal to 0.5% of the area of the adhesion portion with the part of living body by the external stimuli, the resin composition for a cosmetic can be easily peeled off from the living body during the application of the external stimuli. The solidified matter of the resin composition for a cosmetic in the present exemplary embodiment may undergo the volume change in more than or equal to 0.9% of the area of the adhesion portion with the part of living body by the application of the external stimuli. Since the solidified matter of the resin composition for a cosmetic undergoes the volume change in more than or equal to 0.9% of the area of the adhesion portion with the part of living body by the external stimuli, the resin composition for a cosmetic can be more easily peeled off from the living body during the application of the external stimuli. The solidified matter of the resin composition for a cosmetic in the present exemplary embodiment may undergo the volume change in 100% of the area of the adhesion portion with the living body by the external stimuli.

The resin composition for a cosmetic undergoing the volume change in an area of more than or equal to 0.5% or more than or equal to 0.9% of the adhesion portion can be obtained, for example, by adjusting the content ratio of the stimulus-responsive material in the resin composition for a cosmetic. For example, by dissolving or uniformly dispersing the stimulus-responsive material in the resin composition for a cosmetic at a ratio of more than or equal to 0.5 vol % or more than or equal to 0.9 vol %, it is possible to prepare the resin composition for a cosmetic undergoing the volume change in the area as described above. The content ratio of the stimulus-responsive material in the resin composition for a cosmetic can be confirmed by, for example, NMR, IR, MS, SIMS, ICP, SEM, SEM-EDX, liquid chromatography, or spectrophotometry as described above. Whether or not the stimulus-responsive material is dissolved or uniformly dispersed can be confirmed by the transmission uniformity of the light. It is also possible to use the refractive index or the XRD in the case of the material having the crystal structure. It is also possible to evaluate the permeability or the solubility of the filter by using the viscosity. The volume change in more than or equal to 0.5% of the area or more than or equal to 0.9% of the area of the adhesion portion can be confirmed by, for example, an X-ray transmission method, a ultrasonic inspection method, an optical microscope, or a CCD camera.

The resin composition for a cosmetic in the present exemplary embodiment may include materials other than the stimulus-responsive material. For example, the resin composition for a cosmetic in the present exemplary embodiment may include a polymer material as a base material as another material. The other materials included in the resin composition for a cosmetic in the present exemplary embodiment are selected from, for example, materials having high biocompatibility. For example, the polymer material as the base material may include an acrylic material, a cyanoacrylate-based material, a silicone-based material, a urethane-based material, a urethane(meth)acrylate-based material, a styrene-based material, an epoxy-based material, and an elastomer, having the high biocompatibility. One of these materials may be included, or a plurality of these materials may be mixed and included. In particular, the urethane(meth)acrylate-based material is a main material of a soak off gel nail, and is excellent from a viewpoint of the adhesive property to nails, safety, and flexibility. Furthermore, the cyanoacrylate-based material is high in biological safety and adhesive force to lashes or the like. Therefore, in a case where the resin composition for a cosmetic in the present exemplary embodiment is used for a cosmetic to adhere to the lashes, the cyanoacrylate-based material can be suitably used.

Furthermore, the resin composition for a cosmetic in the present exemplary embodiment is a resin composition substantially free of an additive having high toxicity to the living body. Examples of the material having the high toxicity to the living body include benzene, ethylene dichloride, acetylene dichloride, monochlorobenzene, ethylbenzene, styrene, lead, mercury, and arsenic. A contents of these materials in the resin composition for a cosmetic in the present exemplary embodiment is, for example, less than or equal to 10 ppm or less, or less than or equal to a detection limit.

The resin composition for a cosmetic in the present exemplary embodiment may have a glass transition point of, for example, less than or equal to 150° C. In a case where the solidified matter undergoes the volume change, the resin composition for a cosmetic being soft to some extent and having the glass transition point of less than or equal to 150° C. obtains an effect of reducing a phenomenon that a large part of the solidified matter is cracked, and easily peeling off the solidified matter from the living body.

The resin composition for a cosmetic in the present exemplary embodiment can reduce the strength of adhesion of the solidified matter of the resin composition for a cosmetic to the part of living body to, for example, less than or equal to 50% with respect to the strength of adhesion before the application of the external stimuli by the application of the external stimuli. Since the strength of adhesion is reduced to less than or equal to 50%, the resin composition for a cosmetic can be more easily peeled off from the living body by applying the external stimuli. The strength of adhesion may be reduced to less than or equal to 30% or less than or equal to 10%. As the strength of adhesion obtained by applying the external stimuli is lower than the strength of adhesion before the application of the external stimuli, that is, initial strength of adhesion, both firm adhesion to the living body before the application of the external stimuli and easy peeling after the application of the external stimuli are possible.

The solidified matter of the resin composition for a cosmetic in the present exemplary embodiment undergoes the volume change by the application of the external stimuli to reduce the strength of adhesion to the part of living body. Such volume change of the solidified matter of the resin composition for a cosmetic occurs, since the stimulus-responsive material undergoes the volume change by the application of the external stimuli. A mechanism allowing the stimulus-responsive material to undergo the volume change by the application of the external stimuli is not particularly limited. The stimulus-responsive material undergoes the volume change by, for example, causing at least one selected from the group consisting of the change in physical property and the chemical reaction by the external stimuli. Examples of the change in physical property include the vaporization, the sublimation, the change in density, the liquefaction, and the change in viscoelasticity, as described above. Examples of the chemical reaction include the reaction accompanied with the change in molecular structure and the decomposition reaction, as described above. For example, the stimulus-responsive material may undergo the volume change due to a chemical reaction caused by the external stimuli or generation of a gas caused by the sublimation or the like. The volume change of the stimulus-responsive material caused by the external stimuli may be, for example, a volume change of more than or equal to 10%, a volume change of more than or equal to 20%, or a volume change of more than or equal to 50%. Since the volume change of more than or equal to 10% occurs, the strength of adhesion easily decreases during the application of the external stimuli. Since the volume change of more than or equal to 20% occurs, the strength of adhesion more easily decrease during the application of the external stimuli. Since the volume change of more than or equal to 50% occurs, an effect of more stably reducing the strength of adhesion is obtained even with a small stimulus.

For example, the stimuli-responsive material may generate a gas by the application of the external stimuli, and therefore, the volume change of the stimulus-responsive material may be caused. Gas generation by the stimulus-responsive material by the application of the external stimuli may, for example, occur by a chemical reaction such as decomposition of the stimulus-responsive material by the application of the external stimuli, or occur by sublimation of the stimulus-responsive material. Such gas generation causes the volume change of the solidified matter of the resin composition for a cosmetic. Specifically, since bubbles are generated in the solidified matter of the resin composition for a cosmetic by the gas to be generated, the volume change of the solidified matter occurs. Since the volume change of the solidified matter by the generation of the bubbles also occurs at an adhesion interface with the living body, the adhesion area between the solidified material and the living body is reduced, and the strength of adhesion decreases.

In a case where the volume change occurs by the gas generation, a volume change rate is large. Therefore, the peeling by the application of the external stimuli becomes easier. Examples of the gas to be generated include nitrogen, oxygen, hydrogen, carbon dioxide, water, ammonia, carbon monoxide, nitric acid, and formaldehyde. In particular, in the case of the nitrogen, the carbon dioxide, and the water, the resin composition for a cosmetic is safer since it is excellent in biological safety and is free of flammability.

The stimulus-responsive material may generate a liquid or a supercritical fluid by the application of the external stimuli, and therefore, the volume change of the stimulus-responsive material may be caused. The generation of such liquid or supercritical fluid causes the volume change of the solidified matter of the resin composition for a cosmetic. Specifically, for example, since the generation of the liquid or the supercritical fluid is generated at an adhesion portion between the solidified matter and the living body causes the volume change of the solidified matter at the adhesion interface with the living body, the strength of adhesion of the solidified matter to the living body is reduced.

Furthermore, the application of the external stimuli causes expansion of the stimulus-responsive material, and as a result, the solidified matter of the resin composition for a cosmetic may undergo the volume change. The strength of adhesion of the solidified matter to the living body is also reduced due to the volume change caused by expansion of such solidified matter.

Furthermore, the stimulus-responsive material may shrink by the application of the external stimuli, and as a result, the solidified matter of the resin composition for a cosmetic may undergo the volume change. As the volume of the solidified matter of the resin composition for a cosmetic decreases due to the shrinkage of the stimulus-responsive material, the strength of adhesion of the solidified matter to the living body is reduced. Examples of the stimulus-responsive material to shrink by the application of the external stimuli include a material that crosslinks by the application of the light or the heat or a material that is being crystallized.

In a case where the resin composition for a cosmetic in the present exemplary embodiment is disposed on the living body, a thickness of the resin composition for a cosmetic may be, for example, more than or equal to 10 μm. Since the thickness of the resin composition for a cosmetic is more than or equal to 10 μm, sufficient strength of adhesion with the living body is obtained, and in a case where the gas is generated by the application of the external stimuli, the gas is less likely to escape in a surface direction, and a larger amount of the gas can be retained between the living body and the solidified matter of the resin composition for a cosmetic. Furthermore, in a case where another cosmetic is further disposed on the disposed resin composition for a cosmetic, a sum of the thickness of the resin composition for a cosmetic and a thickness of the another cosmetic disposed on the resin composition for a cosmetic may be, for example, more than or equal to 10 μm. In a case where the sum of the thickness of the resin composition for a cosmetic and the thickness of the another cosmetic composition to be disposed on the resin composition for a cosmetic of more than or equal to 10 μm generates the gas by the application of the external stimuli, the gas is less likely to escape in the surface direction, and the larger amount of the gas can be retained between the living body and the solidified matter of the resin composition for a cosmetic. Accordingly, it is possible to more easily peel off the resin composition for a cosmetic from the living body during the application of the external stimuli.

In a case where the resin composition for a cosmetic in the present exemplary embodiment causes the volume change of the solidified matter of the resin composition for a cosmetic by the gas generation due to the stimulus-responsive material, the solidified matter of the resin composition for a cosmetic may have voids inside. For example, when a plurality of fine voids are present in the solidified matter of the resin composition for a cosmetic, it is considered that the gas easily gathers, since a movement resistance of a void portion is smaller than a movement resistance of a bulk portion (that is, a portion other than the void portion of the resin composition for a cosmetic) in a case where the gas is generated during the application of the external stimuli. Gathering of a large amount of the gas in the void portion causes a larger volume change, and an effect that the solidified matter of the resin composition for a cosmetic is easily peeled off from the living body is obtained. A diameter of the void may be, for example, less than or equal to 50 μm. A plurality of the voids may be present in the solidified matter of the resin composition for a cosmetic. Since many smaller voids (for example, the void having a diameter of less than or equal to 10 μm) are present, it is possible to achieve a large adhesion area with the living body while retaining many voids. Therefore, with such configuration, both stronger adhesion to the living body before the application of the external stimuli and an easier peeling property after the application of the external stimuli can be achieved. Examples of a method for providing the voids in the solidified matter of the resin composition for a cosmetic include a method for including a gas such as air, nitrogen, or carbon dioxide in the resin composition for a cosmetic, and a method for including a porous material having nanopores or micropores in the resin composition for a cosmetic in a state where the gas such as air is included in the pores. Furthermore, when the resin composition for a cosmetic is solidified on the living body, the void can also be provided by generating the gas or the like.

Since the resin composition for a cosmetic in the present exemplary embodiment has a glass transition point of, for example, less than or equal to 150° C., when the gas is generated by the external stimuli, the resin composition for a cosmetic is likely to expand, and the larger volume change occurs. Accordingly, easier peeling becomes possible.

In the resin composition for a cosmetic in the present exemplary embodiment, in a case where the volume change of the solidified matter of the resin composition for a cosmetic by the gas generation due to the stimulus-responsive material occurs, the stimulus-responsive material in the resin composition for a cosmetic in the present exemplary embodiment may include the azide compound. The azide compound may be a glycidyl azide polymer. The glycidyl azide polymer has a property of decomposing and releasing the nitrogen gas in a case of irradiation with light in a wavelength region of, for example, about less than or equal to 400 nm. Therefore, in a case where the resin composition for cosmetic in the present exemplary embodiment includes the glycidyl azide polymer, the resin composition for a cosmetic has a high peeling property during the application of the stimulus. Furthermore, the light having the wavelength between 280 nm and 365 nm (inclusive) can be used as the external stimuli. The light in such wavelength range is excellent in safety to the living body. Furthermore, the stimulus-responsive material in response to the light in such wavelength range can prevent the strength of adhesion of the resin composition for a cosmetic to the living body from being reduced in the normal living environment. In addition, since the light in such wavelength range can be used as the external stimulus, the effective peeling at any timing can be achieved by a simpler method.

In a case where the stimulus-responsive material includes the glycidyl azide polymer, the resin composition for a cosmetic in the present exemplary embodiment may include the glycidyl azide polymer ranging from, for example, more than or equal to 0.5 mass % to less than or equal to 50 mass %. Since the glycidyl azide polymer is included in an amount of more than or equal to 0.5 mass %, the resin composition for a cosmetic in the present exemplary embodiment can improve the peeling property from the living body during the application of the external stimuli. Since the glycidyl azide polymer is included in an amount of less than or equal to 50 mass %, an effect of easily maintaining physical properties of the polymer material or the like as the base material in the resin composition for a cosmetic is obtained. Therefore, for example, an effect of improving the strength of adhesion of the solidified matter of the resin composition for a cosmetic to the living body before the application of the external stimuli is obtained. A concentration of the glycidyl azide polymer in the resin composition for a cosmetic may be more than or equal to 0.9 mass %, more than or equal to 1 mass %, or more than or equal to 2 mass %. Since the concentration of the glycidyl azide polymer in the resin composition for a cosmetic is more than or equal to 0.9 mass %, more than or equal to 1 mass %, or more than or equal to 2 mass %, the resin composition for a cosmetic in the present exemplary embodiment can further improve the peeling property from the living body during the application of the external stimuli. Furthermore, the concentration of the glycidyl azide polymer in the resin composition for a cosmetic may be less than or equal to 35 mass %. Since the concentration of the glycidyl azide polymer in the resin composition for a cosmetic is less than or equal to 35 mass %, the resin composition for a cosmetic in the present exemplary embodiment can obtain both properties of a high adhesion before the application of the external stimuli and an excellent peeling property during the application of the external stimuli.

The part of living body that is adhered with the resin composition for a cosmetic in the present exemplary embodiment may be at least one selected from the group consisting of the nail, the lash, and the skin.

The part of living body that is adhered with the resin composition for a cosmetic in the present exemplary embodiment may be at least one selected from the group consisting of the nail and the lash. The nail or the lash does not have a thin, layered stratum corneum structure that is easily peeled off like the skin. Therefore, by disposing the resin composition for a cosmetic in the present exemplary embodiment after a degreasing treatment on the surface is performed, it is possible to obtain strong strength of adhesion capable of causing the cosmetic such as the nail or the eyelash extension including the resin composition for a cosmetic in the present exemplary embodiment to be continuously disposed on the living body for several weeks.

In a case where the part of living body that is adhered with the resin composition for a cosmetic in the present exemplary embodiment is the nail, the resin composition for a cosmetic in the present exemplary embodiment may be, for example, a resin composition for a nail that includes a base coat agent for a nail such as a base coat agent for a gel nail, an adhesive for allowing a nail tip to adhere to the own nail, or a nail agent such as a gel nail agent. In other words, the resin composition for a nail in the present disclosure may include the resin composition for a cosmetic in the present exemplary embodiment. In this case, the resin composition for a nail of the present disclosure may include a material included in a known resin composition for a nail as a material other than the stimulus-responsive material by appropriate selection in accordance with a use.

The resin composition for a cosmetic in the present exemplary embodiment is, in particular, suitably used as the base coat agent for a gel nail. As described in the section of BACKGROUND ART, since the gel nail firmly adheres to the own nail by using, for example, a photocurable resin, a solvent or polishing is required when the gel nail is peeled off from the nail, and the damage to the own nail is very large. However, by using the resin composition for a cosmetic according to the present exemplary embodiment as the base coat agent for a gel nail, the gel nail can firmly adhere to the own nail for a necessary period, and by applying the external stimuli when the peeling is desirable, the damage to the own nail can be easily suppressed, and the gel nail and the base coat can be easily peeled off from the own nail. Therefore, the base coat agent for a gel nail of the present disclosure may include the resin composition for a cosmetic according to the present exemplary embodiment. In this case, the base coat agent for a gel nail of the present disclosure may include a material included in a known base coat agent for a gel nail as a material other than the stimulus-responsive material by appropriate selection.

Hereinafter, an example that the resin composition for a cosmetic according to the present exemplary embodiment is used as the base coat agent for a gel nail will be described.

FIG. 1 is a schematic sectional view illustrating a state where a base coat and a gel nail adhere to a nail when a resin composition for a cosmetic in the present exemplary embodiment is used as a base coating agent for a gel nail. As illustrated in FIG. 1, base coat 2 adheres onto nail 1, and gel nail 3 adheres onto base coat 2. Base coat 2 is formed by solidifying the resin composition for a cosmetic. FIG. 2 is a schematic sectional view illustrating a state where a base coat and a gel nail including a color gel nail and a top gel nail adhere to a nail when a resin composition for a cosmetic in the present exemplary embodiment is used as a base coat agent for a gel nail. As illustrated in FIG. 2, gel nail 3 may have a two-layer structure including color gel 3a and top gel 3b. In FIG. 2, gel nail 3 has the two-layer structure, but gel nail 3 may have, for example, a multilayer structure of more than or equal to three layers including a plurality of color gel layers.

FIG. 3 is a schematic sectional view illustrating a state where a base coat and a gel nail adhere to a nail via a thin membrane layer when a resin composition for a cosmetic in the exemplary embodiment is used as the base coat agent for a gel nail. As illustrated in FIG. 3, base coat 2 including the resin composition for a cosmetic in the present exemplary embodiment may adhere to nail 1 via thin membrane layer 4. Thin membrane layer 4 directly adhering to nail 1 usually has a physical property such as a water contact angle close to the physical property of nail 1. Therefore, base coat 2 including the resin composition for a cosmetic in the present exemplary embodiment can firmly adhere to nail 1 via thin membrane layer 4. Thin membrane layer 4 may be, for example, a primer or a base gel. Thin membrane layer 4 may be a base coat including a material different from base coat 2 including the resin composition for a cosmetic in the present exemplary embodiment.

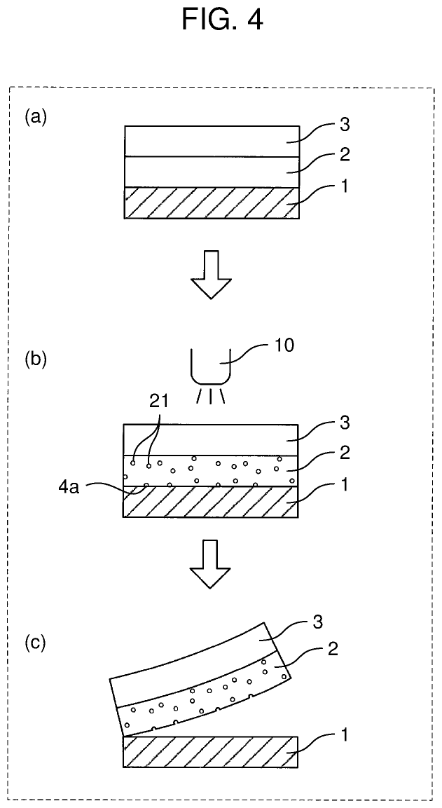

FIG. 4 is a schematic sectional view explaining an example of a principle of a decrease in strength of adhesion of base coat 2 to nail 1 when a resin composition for a cosmetic in the present exemplary embodiment is used as the base coat agent for a gel nail. Here, a case where the stimulus-responsive material included in the resin composition for a cosmetic is a material that generates the gas by the external stimuli will be described as an example. As illustrated in part (a) of FIG. 4, before the external stimuli is applied, base coat 2 firmly adheres to a surface of nail 1, and gel nail 3 adheres onto base coat 2. As illustrated in part (b) of FIG. 4, when the external stimuli is applied to base coat 2 by stimulus generator 10, the stimulus-responsive material included in base coat 2 generates the gas, bubbles 21 are generated in base coat 2, and a volume change of the base coat occurs. Bubbles 21 are also generated at interface 4a between base coat 2 and nail 1, and also cause the volume change of base coat 2 at an adhesion portion with nail 1, and as a result, a substantial contact area between base coat 2 and nail 1 is reduced. Accordingly, as illustrated in part (c) of FIG. 4, the strength of adhesion of base coat 2 to nail 1 is reduced, and base coat 2 and gel nail 3 are easily removed from nail 1. Note that when nail 1 and base coat 2 are peeled off at the interface, base coat 2 and gel nail 3 may be peeled off at the interface at the same time.

In a case where the part of living body that is adhered with the resin composition for a cosmetic in the present exemplary embodiment is a lash, the resin composition for a cosmetic in the present exemplary embodiment may be, for example, an adhesive for an eyelash extension. In other words, the adhesive for an eyelash extension in the present disclosure may include the resin composition for a cosmetic according to the present exemplary embodiment. In this case, the adhesive for an eyelash extension of the present disclosure can include a material included in a known adhesive for an eyelash extension as a material other than the stimulus-responsive material by appropriate selection.

[1-2. Effects or the Like]

As described above, in the present exemplary embodiment, the resin composition in a cosmetic is a resin composition for a cosmetic used for a cosmetic to be adhered to the part of living body, and the resin composition for a cosmetic includes the stimulus-responsive material undergoing the volume change by external stimuli, the external stimuli excluding both an external stimulus due to contact with a solvent and an external stimulus due to contact with a solution. The solidified matter of the resin composition for a cosmetic has a surface with the water contact angle between 30° and 110° (inclusive), and the solidified matter of the resin composition for a cosmetic undergoes the volume change by the application of the external stimuli to reduce the strength of adhesion of the part of living body to the resin composition for a cosmetic.

According to such configuration, the resin composition for a cosmetic in the present exemplary embodiment can firmly adhere to the part of living body, and can be easily peeled off from the living body at any timing.

In the present exemplary embodiment, the external stimulus may be at least one selected from the group consisting of light, heat, magnetic force, electricity, and external force. According to such configuration, the peeling of the resin composition for a cosmetic in the present exemplary embodiment from the living body becomes easier, and the direct damage to the living body is less likely to occur during the peeling.

In the present exemplary embodiment, the external stimulus may be at least one selected from the group consisting of the light and the heat. According to such configuration, the treatment of peeling off the resin composition for a cosmetic from the living body becomes easier, and the direct damage to the living body can be further suppressed.

In the present exemplary embodiment, the external stimulus may be the light having the wavelength ranging from 280 nm up to, not including 400 nm. With such configuration, it is possible to implement the effective peeling at any timing by a simpler method and to further suppress the damage to the living body while preventing a decrease in strength of adhesion of the resin composition for a cosmetic to the living body in the normal living environment.

In the present exemplary embodiment, the solidified matter of the resin composition for a cosmetic may have the transmittance of more than or equal to 10% for the light having any wavelength ranging from 280 nm up to, not including 400 nm. Furthermore, the solidified matter of the resin composition for a cosmetic may have the transmittance of more than or equal to 10% for the light having any wavelength between 280 nm and 365 nm (inclusive). Furthermore, the solidified matter of the resin composition for a cosmetic may have the transmittance of more than or equal to 10% for the light having any wavelength between 280 nm and 350 nm (inclusive). According to such configuration, the resin composition for a cosmetic in the present exemplary embodiment can be effectively peeled off from the living body.

In the present exemplary embodiment, the resin composition for a cosmetic may include more than or equal to 0.5 vol % of the stimulus-responsive material. According to such configuration, when the external stimuli are applied, the strength of adhesion of the solidified matter of the resin composition for a cosmetic to the living body can be effectively reduced, and the resin composition for a cosmetic can be more easily peeled off from the living body.

In the present exemplary embodiment, the resin composition for a cosmetic may include more than or equal to 0.9 vol % of the stimulus-responsive material. According to such configuration, when the external stimuli are applied, the strength of adhesion of the solidified matter of the resin composition for a cosmetic to the living body can be more effectively reduced, and the resin composition for a cosmetic can be still more easily peeled off from the living body.

In the present exemplary embodiment, the solidified matter of the resin composition for a cosmetic may undergo the volume change in more than or equal to 0.5% of the area of the adhesion portion with the part of living body by the application of the external stimuli. According to such configuration, the resin composition for a cosmetic can be easily peeled off from the living body during the application of the external stimuli.

In the present exemplary embodiment, the solidified matter of the resin composition for a cosmetic may undergo the volume change in more than or equal to 0.9% of the area of the adhesion portion with the part of living body by the application of the external stimuli. According to such configuration, the resin composition for a cosmetic can be more easily peeled off from the living body during the application of the external stimuli.

In the present exemplary embodiment, the strength of adhesion of the solidified matter of the resin composition for a cosmetic to the part of living body may be reduced to less than or equal to 50% with respect to the strength of adhesion before the application of the external stimuli by applying the external stimuli. According to such configuration, the resin composition for a cosmetic can be more easily peeled off from the living body.

In the present exemplary embodiment, the solidified matter of the resin composition for a cosmetic may undergo the volume change by generating the gas in the adhesion portion with the part of living body by the application of the external stimuli. According to such configuration, since the volume change rate caused by the application of the external stimuli is large, the resin composition for a cosmetic is more easily peeled off by the application of the external stimuli.

In the present exemplary embodiment, the stimulus-responsive material may include the azide compound. According to such configuration, the resin composition for a cosmetic has the high peeling property during the application of the stimulus, and can also be excellent in safety.

In the present exemplary embodiment, the stimulus-responsive material may include the glycidyl azide polymer. According to such configuration, the resin composition for a cosmetic has the high peeling property during the application of the stimulus, and can also be excellent in safety.

In the present exemplary embodiment, the resin composition for a cosmetic may include the glycidyl azide polymer ranging from more than and equal to 0.5 mass % to less than or equal to 50 mass %. According to such configuration, the resin composition for a cosmetic can improve the peeling property from the living body during the application of the external stimuli.

In the present exemplary embodiment, the part of living body may be at least one selected from the group consisting of the nail and the lash. According to such configuration, by disposing the resin composition for a cosmetic after the degreasing treatment on the surface of the living body, it is possible to obtain the strong strength of adhesion capable of causing the cosmetic such as the nail or the eyelash extension including the resin composition for a cosmetic to be continuously disposed on the living body for several weeks.

In the present exemplary embodiment, the resin composition for a nail includes the resin composition for a cosmetic in the present exemplary embodiment. With the resin composition for a nail having such configuration, the nail can firmly adhere to the own nail for a necessary period, and by applying the external stimuli when the peeling is desirable, the damage to the own nail can be easily suppressed, and the nail can be easily peeled off from the own nail.

In the present exemplary embodiment, the base coat agent for a gel nail includes the resin composition for a cosmetic in the present exemplary embodiment. With the base coat agent for a gel nail having such configuration, the gel nail can firmly adhere to the own nail for a necessary period, and by applying the external stimuli when the peeling is desirable, the damage to the own nail can be easily suppressed, and the gel nail and the base coat can be easily peeled off from the own nail.

In the present exemplary embodiment, the adhesive for an eyelash extension includes the resin composition for a cosmetic in the present exemplary embodiment. With the adhesive for an eyelash extension having such configuration, the eyelash extension can firmly adhere to the eyelash for a necessary period, and by applying the external stimulus when the peeling is desirable, the damage to the eyelash can be easily suppressed, and the eyelash extension can be easily peeled off.

### Other Exemplary Embodiments

As described above, the exemplary embodiment has been described as an example of techniques disclosed in the present application. However, the techniques of the present disclosure are not limited, but may be applicable to exemplary embodiments with changes, additions, omissions, or the like. Furthermore, respective components described in the above exemplary embodiments may be combined to make a new exemplary embodiment.

Therefore, other exemplary embodiments will be described below.

The resin composition for a cosmetic in the present disclosure is a resin composition for a cosmetic used in the cosmetic to be adhered to the part of living body, the resin composition for a cosmetic may include the stimulus-responsive material that undergoes the volume change by external stimuli, the external stimuli excluding both an external stimulus due to contact with a solvent and an external stimulus due to contact with a solution, and the solidified matter of the resin composition for a cosmetic may undergo volumetric increment and decrement by the application of the external stimuli to reduce the strength of adhesion to the part of living body.

The resin composition for a cosmetic in the present exemplary embodiment may be capable of increasing the strength of adhesion to the living body by the external stimuli. For example, the resin composition for a cosmetic in the present exemplary embodiment may improve the strength of adhesion to the living body by the application of the external stimuli different from external stimuli for reducing the strength of adhesion to the living body. The external stimuli different from the external stimuli for reducing the strength of adhesion may be those different in type from the external stimuli for reducing the strength of adhesion, or may be external stimuli of the same type but under different conditions (for example, a wavelength of the light, an intensity of the light, or a temperature of the heat).

Note that the exemplary embodiment described above is to exemplify the techniques in the present disclosure, and thus, various modifications, replacements, additions, omissions, and the like can be made in the scope of claims or in an equivalent scope of the claims.

### EXAMPLES

The resin composition for a cosmetic of the present disclosure will be described in more detail with reference to examples. The resin composition for a cosmetic of the present disclosure is not limited to the following examples.

[Evaluation of Rate of Change in Strength of Adhesion Before and After Irradiation with Ultraviolet Light]

### Example 1

A glycidyl azide polymer (GAP5003 manufactured by NOF CORPORATION) was used as the stimulus-responsive material that undergoes the volume change by application of ultraviolet light. The glycidyl azide polymer has a property of releasing the nitrogen gas by the irradiation with the light in the wavelength region of about less than or equal to 400 nm.

The glycidyl azide polymer was added to an acrylic polymer (Acridic manufactured by DIC Corporation), a solid content concentration being 0.99 mass % and sufficiently mixed to be uniform to prepare the resin composition for a cosmetic. A fact that the acrylic polymer and the glycidyl azide polymer were sufficiently mixed with each other to be uniform was confirmed in accordance with a fact that a transmission state of the light was uniform in the resin composition for a cosmetic obtained. Since a density of the glycidyl azide polymer is 1.3 g/cm³ and a density of the acrylic polymer is generally 1.2 g/cm³, a volume ratio of 0.99 mass % of the glycidyl azide polymer is 0.91 vol %. It is considered that the glycidyl azide polymer is present in 0.91% of the area of the adhesion portion with an object to be adhered in the resin composition for a cosmetic of the present example uniformly mixed. In other words, the solidified matter of the resin composition for a cosmetic of the present example undergoes the volume change by generating the nitrogen gas in 0.91% of an area of the adhesion portion with the object to be adhered.

For evaluation of the strength of adhesion, a polyethylene terephthalate (PET) film was used that has a value close to a water contact angle with respect to a surface of the nail in a case where the nail was degreased. The water contact angle with respect to the surface of the nail in a case where the nail was degreased was approximately 60° to 75°, whereas a contact angle of the water on the PET film used for evaluation was 62°. In general, in a case where a nail is applied to the nail, the nail is degreased to improve the adhesive property with the fingernail and nail. Therefore, by using a PET film having a contact angle close to the contact angle of the nail degreased, it is considered that an evaluation close to the strength of adhesion to the nail can be made.

The resin composition for a cosmetic of the present example was applied to the PET film by performing gap coating with a thickness of 50 μm, and then sufficiently dried at room temperature to be solidified. The strength of adhesion in a case where the solidified matter of the resin composition for a cosmetic having a size of 10×15 mm was peeled off from the PET film at a speed of 20 mm/min was measured with a digital force gauge (ZTA manufactured by IMADA Co., Ltd.).

In addition, another sample prepared in a similar manner was irradiated with the ultraviolet light at 1.8 J/cm². Black light (FL4BLB/N manufactured by Toshiba Lighting & Technology Corporation, with an intensity peak wavelength of about 350 nm) was used as the ultraviolet light. An intensity of the ultraviolet light was measured by using a UV light mater (UV-37SD manufactured by CUSTOM corporation), and an irradiation amount of the ultraviolet light is set to 1.8 J/cm² by adjusting irradiation time. The strength of adhesion ratio after the irradiation with the ultraviolet light at 1.8 J/cm² was 37.6% in a case where the strength of adhesion ratio before the irradiation with the ultraviolet light was 100%.

### Example 2

The strength of adhesion of the solidified matter of the resin composition for a cosmetic before and after the irradiation with the ultraviolet light was evaluated by an evaluation method similar to the evaluation method in Example 1 except that the strength of adhesion was evaluated after the irradiation with the ultraviolet light at 3.0 J/cm² by using the resin composition for a cosmetic prepared by the same method as in Example 1. The strength of adhesion ratio after the irradiation with the ultraviolet light was 39.6% in a case where the strength of adhesion ratio before the irradiation with the ultraviolet light was 100%.

### Example 3

The resin composition for a cosmetic was obtained in a manner similar to Example 1, except that the glycidyl azide polymer was added, the solid content concentration being 2.5 mass %. A fact that the acrylic polymer and the glycidyl azide polymer were sufficiently mixed with each other to be uniform was confirmed in accordance with a fact that a transmission state of the light was uniform in the resin composition for a cosmetic obtained. The volume ratio of the glycidyl azide polymer is 2.3 vol %. It is considered that the glycidyl azide polymer is present in 2.3% of the area of the adhesion portion with the object to be adhered in the resin composition for a cosmetic of the present example uniformly mixed. In other words, the solidified matter of the resin composition for a cosmetic of the present example undergoes the volume change by generating the nitrogen gas in 2.3% of the area of the adhesion portion with the object to be adhered.

The strength of adhesion of the solidified matter of the resin composition for a cosmetic before and after the irradiation with the ultraviolet light was evaluated by an evaluation method similar to the evaluation method in Example 1. The strength of adhesion ratio after the irradiation with the ultraviolet light was 23.9% in a case where the strength of adhesion ratio before the irradiation with the ultraviolet light was 100%.

### Example 4

The resin composition for a cosmetic was obtained in a manner similar to Example 1, except that the glycidyl azide polymer was added, the solid content concentration being 38.5 mass %. A fact that the acrylic polymer and the glycidyl azide polymer were sufficiently mixed with each other to be uniform was confirmed in accordance with a fact that a transmission state of the light was uniform in the resin composition for a cosmetic obtained. The volume ratio of the glycidyl azide polymer is 36.6 vol %. It is considered that the glycidyl azide polymer is present in 36.6% of the area of the adhesion portion with the object to be adhered in the resin composition for a cosmetic of the present example uniformly mixed. In other words, the solidified matter of the resin composition for a cosmetic of the present example undergoes the volume change by generating the nitrogen gas in 36.6% of the area of the adhesion portion with the object to be adhered.

After the resin composition for a cosmetic of Example 4 was dried and solidified, the transmittance of the solidified matter of the resin composition for a cosmetic for light having a wavelength of 350 nm was 77.7%. The transmittance of the solidified matter of the resin composition for a cosmetic for the light having the wavelength of 350 nm was measured by using a spectrophotometer (V-770 manufactured by JASCO Corporation). Note that the solidified matter of the resin composition for a cosmetic for transmittance measurement was prepared as follows. First, the resin composition for a cosmetic of Example 4 was subjected to the gap coating with the thickness of 50 μm on a slide glass, and solidified by being dried, and a coating membrane as the solidified matter of the resin composition for a cosmetic was disposed on the slide glass. A transmittance of the coating membrane of the resin composition for a cosmetic thus prepared on the slide glass was measured.

The strength of adhesion of the solidified matter of the resin composition for a cosmetic before and after the irradiation with the ultraviolet light was evaluated by an evaluation method similar to the evaluation method in Example 1. The strength of adhesion ratio after the irradiation with the ultraviolet light was 24.7% in a case where the strength of adhesion ratio before the irradiation with the ultraviolet light was 100%.

### Example 5

The strength of adhesion of the solidified matter of the resin composition for a cosmetic before and after the irradiation with the ultraviolet light was evaluated by an evaluation method similar to the evaluation method in Example 4 except that the strength of adhesion was evaluated after the irradiation with the ultraviolet light at 3.0 J/cm² by using the resin composition for a cosmetic prepared by the same method as in Example 1. The strength of adhesion ratio after the irradiation with the ultraviolet light was 29.7% in a case where the strength of adhesion ratio before the irradiation with the ultraviolet light was 100%.

### Example 6

The resin composition for a cosmetic was obtained in a manner similar to Example 1, except that the glycidyl azide polymer was added, the solid content concentration being 51.7 mass %. A fact that the acrylic polymer and the glycidyl azide polymer were sufficiently mixed with each other to be uniform was confirmed in accordance with a fact that a transmission state of the light was uniform in the resin composition for a cosmetic obtained. The volume ratio of the glycidyl azide polymer is 49.7 vol %. It is considered that the glycidyl azide polymer is present in 49.7% of the area of the adhesion portion with the object to be adhered in the resin composition for a cosmetic of the present example uniformly mixed. In other words, the solidified matter of the resin composition for a cosmetic of the present example undergoes the volume change by generating the nitrogen gas in 49.7% of the area of the adhesion portion with the object to be adhered.

After the resin composition for a cosmetic of Example 6 was dried and solidified, the transmittance of the solidified matter of the resin composition for a cosmetic for the light with the wavelength of 350 nm was 72.9%. The transmittance of the solidified matter of the resin composition for a cosmetic for the light having the wavelength of 350 nm was measured by using a spectrophotometer (V-770 manufactured by JASCO Corporation). Note that the solidified matter of the resin composition for a cosmetic for transmittance measurement was prepared as follows. First, the resin composition for a cosmetic of Example 5 was subjected to the gap coating with the thickness of 50 μm on the slide glass, and solidified by being dried, and the coating membrane as the solidified matter of the resin composition for a cosmetic was disposed on the slide glass. A transmittance of the coating membrane of the resin composition for a cosmetic thus prepared on the slide glass was measured.

The strength of adhesion of the solidified matter of the resin composition for a cosmetic before and after the irradiation with the ultraviolet light was evaluated by an evaluation method similar to the evaluation method in Example 1. The strength of adhesion ratio after the irradiation with the ultraviolet light was 46.5% in a case where the strength of adhesion ratio before the irradiation with the ultraviolet light was 100%.

### Comparative Example 1

By using the acrylic polymer (Acridic manufactured by DIC Corporation) without the glycidyl azide polymer added, the strength of adhesion of the acrylic polymer before and after the irradiation with the ultraviolet light was evaluated by an evaluation method similar to the evaluation method in Example 1. The strength of adhesion ratio after the irradiation with the ultraviolet light was 97.8% in a case where the strength of adhesion ratio before the irradiation with the ultraviolet light was 100%.

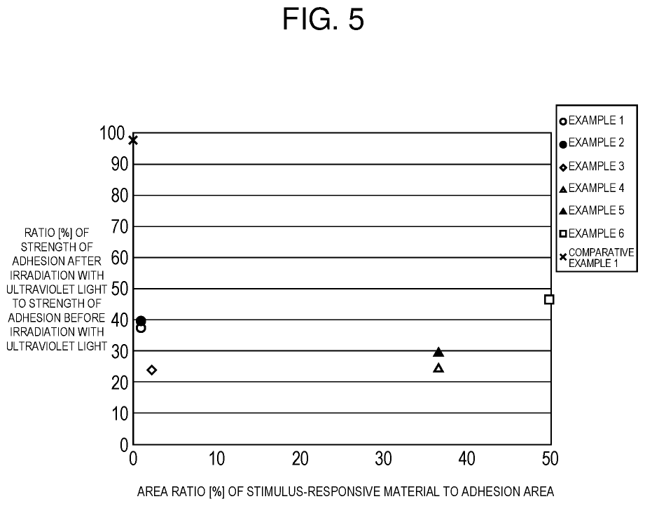

FIG. 5 is a graph illustrating a relationship between an strength of adhesion ratio after irradiation with ultraviolet light in a case where the strength of adhesion before the irradiation with the ultraviolet light is 100% and an area ratio of a stimulus-responsive material to an adhesion area, for a resin composition for a cosmetic in Examples 1 to 6 and an acrylic polymer in Comparative Example 1. From the graph of FIG. 5, it can be seen that the strength of adhesion ratio can be reduced to less than or equal to 50% by the application of the ultraviolet light by disposing the stimulus-responsive material that undergoes the volume change due to gasification in more than or equal to 0.91% of the area of the adhesion portion.

[Evaluation of Adhesive Property to Nail]

### Example 7

The resin composition for a cosmetic prepared by a method similar to the method in Example 1 was used to be applied to entire surfaces of nails of a human hand with a brush for a nail, and the resin composition for a cosmetic was disposed on the nails. The resin composition for a cosmetic on the nails was sufficiently dried and solidified at room temperature to form the coating membrane as the solidified matter of the resin composition for a cosmetic on the nails. Thereafter, the person went on with his or her normal life. It was confirmed that the coating membrane was not peeled off from the nail for more than or equal to 24 hours.

Furthermore, the resin composition for a cosmetic was applied onto the slide glass by gap coating, and a film-shaped sheet was prepared by sufficiently drying and solidifying the coating membrane at room temperature. The water contact angle with respect to a surface of the film-shaped sheet was evaluated. The contact angle was measured by a droplet method with DROPMASTER DMO-501 manufactured by Kyowa Interface Science Co., Ltd. The contact angle was evaluated by dropping 5 μL of the water. Analysis was performed by a θ/2 method. The contact angle was measured five times to determine an average value. The surface of the film-shaped sheet, that is, the surface of the solidified matter of the resin composition for a cosmetic had a water contact angle of the water of 72.6°.

### Example 8

The resin composition for a cosmetic prepared by a method similar to the method in Example 6 was used to be applied to the entire surfaces of the nails of the human hand with the brush for a nail, and the resin composition for a cosmetic was disposed on the nails. The resin composition for a cosmetic on the nails was sufficiently dried and solidified at room temperature to form the coating membrane as the solidified matter of the resin composition for a cosmetic on the nails. Thereafter, the person went on with his or her normal life. It was confirmed that the coating membrane was not peeled off from the nail for more than or equal to 24 hours. Furthermore, the water contact angle with respect to the surface of the solidified matter of the resin composition for a cosmetic was measured by a method similar to the method in Example 7. The water contact angle was 73.0°.

### Example 9

The resin composition for a cosmetic was obtained in a similar manner as in Example 1 except that a commercially available nail base coat agent “melty gel base coat” was used instead of the acrylic polymer, and the glycidyl azide polymer was added, the solid content concentration being 0.54 mass %. A fact that the melty gel base coat and the glycidyl azide polymer were sufficiently mixed with each other to be uniform was confirmed in accordance with a fact that the transmission state of the light was uniform in the resin composition for a cosmetic obtained. Since the main material of the melty gel base coat is an acrylic-based resin and a density of the acrylic-based resin is generally about 1.2 g/cm³, the volume ratio of the glycidyl azide polymer is 0.5 vol %. It is considered that the glycidyl azide polymer is present in 0.5% of the area of the adhesion portion with the object to be adhered in the resin composition for a cosmetic of the present example uniformly mixed. In other words, the solidified matter of the resin composition for a cosmetic of the present example undergoes the volume change by generating the nitrogen gas in 0.5% of the area of the adhesion portion with the object to be adhered.

The resin composition for a cosmetic of Example 9 was used to be applied to the entire surfaces of the nails of the human hand with the brush for a nail, and the resin composition for a cosmetic was disposed on the nails. The resin composition for a cosmetic on the nails was sufficiently dried and solidified at room temperature to form the coating membrane as the solidified matter of the resin composition for a cosmetic on the nails. Thereafter, the person went on with his or her normal life. It was confirmed that the coating membrane was not peeled off from the nail for more than or equal to 24 hours.

Furthermore, the water contact angle with respect to the surface of the solidified matter of the resin composition for a cosmetic of the present example was measured by a method similar to the method in Example 7. The water contact angle was 67.5°.

### Example 10

The resin composition for a cosmetic was obtained in a similar manner as in Example 1 except that a commercially available nail “integrate top & base coat” was used instead of the acrylic polymer, and the glycidyl azide polymer was added, the solid content concentration being 51.7 mass %. A fact that the integrate top & base coat and the glycidyl azide polymer were sufficiently mixed with each other to be uniform was confirmed in accordance with to the fact that the transmission state of the light was uniform in the resin composition for a cosmetic obtained. Since the main material of the integrate top & base coat is the acrylic-based resin and the density of the acrylic-based resin is generally about 1.2 g/cm³, the volume ratio of the glycidyl azide polymer is 49.7 vol %. It is considered that the glycidyl azide polymer is present in 49.7% of the area of the adhesion portion with the object to be adhered in the resin composition for a cosmetic of the present example uniformly mixed. In other words, the solidified matter of the resin composition for a cosmetic of the present example undergoes the volume change by generating the nitrogen gas in 49.7% of the area of the adhesion portion with the object to be adhered.

The resin composition for a cosmetic of Example 10 was used to be applied to the entire surfaces of the nails of the human hand with the brush for nails, and the resin composition for a cosmetic was disposed on the nails. The resin composition for a cosmetic on the nails was sufficiently dried and solidified at room temperature to form the coating membrane as the solidified matter of the resin composition for a cosmetic on the nails. Thereafter, the person went on with his or her normal life. It was confirmed that the coating membrane was not peeled off from the nail for more than or equal to 24 hours.

Furthermore, the water contact angle with respect to the surface of the solidified matter of the resin composition for a cosmetic of the present example was measured by a method similar to the method in Example 7. The water contact angle was 100.5°.

### Example 11

The resin composition for a cosmetic was obtained in a similar manner as in Example 1 except that a base coat gel nail having a commercially available urethane acrylate polymer as a main material was used instead of the acrylic polymer, and the glycidyl azide polymer was added, the solid content concentration being 20.0 mass %. A fact that the base coat gel nail and the glycidyl azide polymer were sufficiently mixed with each other to be uniform was confirmed in accordance with the fact that the transmission state of the light was uniform in the resin composition for a cosmetic obtained. Since a density of the urethane acrylate polymer as the main material of the base coat gel nail is generally about 1.1 g/cm³, the volume ratio of the glycidyl azide polymer is 17.5 vol %. It is considered that the glycidyl azide polymer is present in 17.5% of the area of the adhesion portion with the object to be adhered in the resin composition for a cosmetic of the present example uniformly mixed. In other words, the solidified matter of the resin composition for a cosmetic of the present example undergoes the volume change by generating the nitrogen gas in 17.5% of the area of the adhesion portion with the object to be adhered.

The resin composition for a cosmetic of Example 11 was used to be applied to the entire surfaces of the nails of the human hand with the brush for a nail, and the resin composition for a cosmetic was disposed on the nails. By using a gel nail curing light to irradiate the resin composition for a cosmetic on the nails with an LED having a wavelength of 390 nm to 400 nm for 2 minutes, the resin composition for a cosmetic on the nails was cured. By wiping off the resin uncured on a surface with ethanol, the coating membrane as the solidified matter of the resin composition for a cosmetic was disposed on the nails. Thereafter, the person went on with his or her normal life. It was confirmed that the coating membrane was not peeled off from the nail for more than or equal to 24 hours.

Furthermore, the water contact angle with respect to the surface of the solidified matter of the resin composition for a cosmetic of the present example was measured by a method similar to the method in Example 7. The water contact angle was 87.9°.

### Example 12

The resin composition for a cosmetic was obtained in the same manner as in Example 1 except that a gel nail prepared by mixing 79.0 mass % of a urethane methacrylate polymer, 20.0 mass % of a quaternary ammonium salt acrylic polymer, and 1.0 mass % of diphenyl(2,4,6-trimethylbenzoyl)phosphine oxide was used instead of the acrylic polymer, and the glycidyl azide polymer was added, the solid concentration being 0.65 mass %. The fact that the gel nail and the glycidyl azide polymer were sufficiently mixed with each other to be uniform was confirmed in accordance with the fact that the transmission state of the light was uniform in the resin composition for a cosmetic obtained. Since a density of the gel nail prepared was 1.0 g/cm³, the volume ratio of the glycidyl azide polymer was 0.5 vol %. It is considered that the glycidyl azide polymer is present in 0.5% of the area of the adhesion portion with the object to be adhered in the resin composition for a cosmetic of the present example uniformly mixed. In other words, the solidified matter of the resin composition for a cosmetic of the present example undergoes the volume change by generating the nitrogen gas in 0.5% of the area of the adhesion portion with the object to be adhered.

The resin composition for a cosmetic of Example 12 was used to be applied to the entire surfaces of the nails of the human hand with the brush for a nail, and the resin composition for a cosmetic was disposed on the nails. By using a gel nail curing light to irradiate the resin composition for a cosmetic on the nails with an LED having a wavelength of 390 nm to 400 nm for 2 minutes, the resin composition for a cosmetic on the nails was cured. By wiping off the resin uncured on a surface with ethanol, the coating membrane as the solidified matter of the resin composition for a cosmetic was disposed on the nails. Thereafter, the person went on with his or her normal life. It was confirmed that the coating membrane was not peeled off from the nail for more than or equal to 24 hours.

Furthermore, the water contact angle with respect to the surface of the solidified matter of the resin composition for a cosmetic of the present example was measured by a method similar to the method in Example 7. The water contact angle was 39.6°.

### Example 13

The resin composition for a cosmetic was obtained in the same manner as in Example 1 except that a gel nail prepared by mixing 99.0 mass % of the urethane methacrylate polymer with 1.0 mass % of the diphenyl(2,4,6-trimethylbenzoyl)phosphine oxide was used instead of the acrylic polymer, and the glycidyl azide polymer was added, the solid concentration being 30.0 mass % with respect to the gel nail prepared. The fact that the gel nail and the glycidyl azide polymer were sufficiently mixed with each other to be uniform was confirmed in accordance with the fact that the transmission state of the light was uniform in the resin composition for a cosmetic obtained. Since the density of the gel nail prepared was 1.1 g/cm³, the volume ratio of the glycidyl azide polymer was 26.6 vol %. It is considered that the glycidyl azide polymer is present in 26.6% of the area of the adhesion portion with the object to be adhered in the resin composition for a cosmetic of the present example uniformly mixed. In other words, the solidified matter of the resin composition for a cosmetic of the present example undergoes the volume change by generating the nitrogen gas in 26.6% of the area of the adhesion portion with the object to be adhered.

The resin composition for a cosmetic of Example 13 was used to be applied to the entire surfaces of the nails of the human hand with the brush for a nail, and the resin composition for a cosmetic was disposed on the nails. By using a gel nail curing light to irradiate the resin composition for a cosmetic on the nails with an LED having a wavelength of 390 nm to 400 nm for 2 minutes, the resin composition for a cosmetic on the nails was cured. By wiping off the resin uncured on a surface with ethanol, the coating membrane as the solidified matter of the resin composition for a cosmetic was disposed on the nails. Thereafter, the person went on with his or her normal life. It was confirmed that the coating membrane was not peeled off from the nail for more than or equal to 24 hours.

Furthermore, the water contact angle with respect to the surface of the solidified matter of the resin composition for a cosmetic of the present example was measured by a method similar to the method in Example 7. The water contact angle was 81.5°.

### Comparative Example 2

The resin composition for a cosmetic was obtained in a similar manner as in Example 1 except that a polyvinyl alcohol adhesive was used instead of the acrylic polymer, and the glycidyl azide polymer was added, the solid content concentration being 0.54 mass %. A fact that the polyvinyl alcohol adhesive and the glycidyl azide polymer were sufficiently mixed with each other to be uniform was confirmed in accordance with the fact that the transmission state of the light was uniform in the resin composition for a cosmetic obtained. Since a density of the polyvinyl alcohol is generally about 1.2 g/cm³, the volume ratio of the glycidyl azide polymer was 0.5 vol %.

The resin composition for a cosmetic of Comparative Example 2 was used to be applied to the entire surfaces of the nails of the human hand with the brush for a nail, and the resin composition for a cosmetic was disposed on the nails. The resin composition for a cosmetic on the nails was sufficiently dried and solidified at room temperature to form the coating membrane as the solidified matter of the resin composition for a cosmetic on the nails. Thereafter, as a result of going on the normal life, the coating membrane was peeled off from the nail within 1 hour.

Furthermore, the water contact angle with respect to the surface of the solidified matter of the resin composition for a cosmetic of the present comparative example was measured by a similar method to the method in Example 7. The water contact angle was 26.8°.

### Comparative Example 3

The resin composition for a cosmetic was obtained in a similar manner as in Example 1 except that a silicone adhesive was used instead of the acrylic polymer, and the glycidyl azide polymer was added, the solid content concentration being 0.65 mass %. The fact that the silicone adhesive and the glycidyl azide polymer were sufficiently mixed with each other to be uniform was confirmed in accordance with the fact that the transmission state of the light was uniform in the resin composition for a cosmetic obtained. Since a density of the silicone is generally about 1.0 g/cm³, the volume ratio of the glycidyl azide polymer was 0.5 vol %.

The resin composition for a cosmetic of Comparative Example 3 was used to be applied to the entire surfaces of the nails of the human hand with the brush for a nail, and the resin composition for a cosmetic was disposed on the nails. The resin composition for a cosmetic on the nails was sufficiently dried and solidified at room temperature to form the coating membrane as the solidified matter of the resin composition for a cosmetic on the nails. Thereafter, as a result of going on the normal life, the coating membrane was peeled off from the nail within 1 hour.

Furthermore, the water contact angle with respect to the surface of the solidified matter of the resin composition for a cosmetic of the present comparative example was measured by a similar method to the method in Example 7. The water contact angle was 110.6°.

As the evaluation of the adhesive property to the nail, after the solidified matter of the resin composition for a cosmetic was disposed on the nail, the normal life went on, and a case where the solidified matter was peeled off within 1 hour was evaluated as x, a case where the solidified matter was peeled off within 1 to 24 hours was evaluated as Δ, and a case where the solidified matter was not peeled off for more than or equal to 24 hours was evaluated as ◯. Furthermore, after the resin composition for a cosmetic was disposed on the nail, the nail was rubbed 100 times with a cotton swab, and the number of times the solidified matter of the resin composition for a cosmetic started to be peeled off from the nail was evaluated. A case where the solidified matter was peeled off within 10 rubs was evaluated as x, a case where the solidified matter was peeled off from 10 to 100 rubs was evaluated as Δ, and a case where the solidified was not peeled off even after 100 rubs was evaluated as ◯. The results of Examples 7 to 13, Comparative Example 2, and Comparative Example 3 are shown in Table 1 below.

TABLE 1

| | Example | Example | Example | Example | Example | Example | Example | Comparative | Comparative |
|---|---|---|---|---|---|---|---|---|---|
| | 7 | 8 | 9 | 10 | 11 | 12 | 13 | Example 2 | Example 3 |
| Contact angle | 72.6° | 73.0° | 67.5° | 100.5° | 87.9° | 39.6° | 81.5° | 26.8° | 110.6° |
| Peeling property | ◯ | ◯ | ◯ | ◯ | ◯ | ◯ | ◯ | X | X |
| for 24 hours | | | | | | | | | |
| Evaluation of | ◯ | ◯ | ◯ | ◯ | ◯ | ◯ | ◯ | X | X |
| rubbing with | (No | (No | (No | (No | (No | (No | (No | (7 times) | (6 times) |
| cotton swab | peeling) | peeling) | peeling) | peeling) | peeling) | peeling) | peeling) | | |
| (number of | | | | | | | | | |
| times of peeling) | | | | | | | | | |

As shown in Table 1, the resin compositions for cosmetics of Examples 7 to 13 exhibited higher adhesive property to the nails as compared with Comparative Example 2 and Comparative Example 3.

[Evaluation of Adhesive Property Before and After Irradiation with Ultraviolet Light]

### Example 14

The coating membrane as the solidified matter of the resin composition for a cosmetic was disposed on the nail chip by applying the resin composition for a cosmetic prepared in a similar manner as in Example 4 to an entire surface of a nail chip (manufactured by Yiwu Runni Art Co., Ltd.) with the brush for a nail, and sufficiently drying and solidifying the resin composition for a cosmetic at room temperature.

### Example 15

The resin composition for a cosmetic prepared in a similar manner as in Example 11 was applied to the entire surface of the nail chip with the brush for a nail and solidified by a similar method to the method in Example 11, and the coating membrane as the solidified matter of the resin composition for a cosmetic was disposed on the nail chip. The resin composition for a cosmetic of Example 11 was cured, and the resin uncured on the surface was wiped off with the ethanol. Accordingly, the coating membrane as the solidified matter of the resin composition for a cosmetic was disposed on the nail chip.

Furthermore, the transmittance of the solidified matter of the resin composition for a cosmetic used in the present example for light having a wavelength of 365 nm was 32.4%. The transmittance of the solidified matter of the resin composition for a cosmetic for the light having the wavelength of 365 nm was measured by using the spectrophotometer (V-770 manufactured by JASCO Corporation). Note that the solidified matter of the resin composition for a cosmetic for transmittance measurement was prepared as follows. First, the resin composition for a cosmetic prepared in a similar manner as in Example 11 was applied onto the slide glass with the brush for a nail in a similar manner as being applied to the nail chip in Example 15 and cured in a similar manner as in Example 11, and the coating membrane as the solidified matter of the resin composition for a cosmetic was disposed on the slide glass. Hereinafter, by wiping off the resin uncured on the surface with the ethanol, the coating membrane as the solidified matter of the resin composition for a cosmetic was disposed on the slide glass. A transmittance of the coating membrane of the resin composition for a cosmetic thus prepared on the slide glass was measured.

### Example 16

The resin composition for a cosmetic prepared in a similar manner as in Example 13 was applied to the entire surface of the nail chip with the brush for a nail and solidified by a similar method to the method in Example 13, and the coating membrane as the solidified matter of the resin composition for a cosmetic was disposed on the nail chip. The resin composition for a cosmetic of Example 13 was cured, and the resin uncured on the surface was wiped off with the ethanol. Accordingly, the coating membrane as the solidified matter of the resin composition for a cosmetic was disposed on the nail chip.

Furthermore, the transmittance of the solidified matter of the resin composition for a cosmetic used in the present example for light having a wavelength of 340 nm was 24.1%. The transmittance of the solidified matter of the resin composition for a cosmetic for the light having the wavelength of 340 nm was measured by using the spectrophotometer (V-770 manufactured by JASCO Corporation). Note that the solidified matter of the resin composition for a cosmetic for transmittance measurement was prepared as follows. First, the resin composition for a cosmetic prepared in a similar manner as in Example 13 was applied onto the slide glass with the brush for a nail in a similar manner as being applied to the nail chip in Example 16 and cured in a similar manner as in Example 13, and the coating membrane as the solidified matter of the resin composition for a cosmetic was disposed on the slide glass. Hereinafter, by wiping off the resin uncured on the surface with the ethanol, the coating membrane as the solidified matter of the resin composition for a cosmetic was disposed on the slide glass. A transmittance of the coating membrane of the resin composition for a cosmetic thus prepared on the slide glass was measured.

### Comparative Example 4

The base coat gel nail was used that is the same as used in Example 11 without addition of the stimulus-responsive material such as the glycidyl azide polymer and has the commercially available urethane acrylate polymer as the main material, and the base coat gel nail was cured in a similar manner as in Example 15 to dispose the gel nail on the nail tip.

### Comparative Example 5

By applying a commercially available silicone-based gluing agent (manufactured by INCREASE HAIR CO., LTD.) without the addition of the stimulus-responsive material such as the glycidyl azide polymer to the entire surface of the nail chip with the brush for a nail, and then sufficiently drying the silicone-based gluing agent at room temperature, a solidified matter of the gluing agent was disposed on the nail chip.

For the coating membrane as, for example, the solidified matter of the resin composition for a cosmetic disposed on the nail tip of Example 14, Example 15, Example 16, Comparative Example 4, and Comparative Example 5, an adhesive property to the nail chip before and after the irradiation with the ultraviolet light was evaluated. A case where the coating membrane on the nail chip was not peeled off even if the coating membrane was rubbed before the irradiation with the ultraviolet light, and polishing as well as use of acetone were required for the peeling was indicated as ◯, and a case where the coating membrane was peeled off when a scratch was made from an end of the nail chip was indicated as x.

In Example 14, Example 15, Comparative Example 4, and Comparative Example 5, the adhesive property of the coating membrane to the nail chip was evaluated after the irradiation with the ultraviolet light at 3.6 J/cm² by using the LED having the wavelength of 365 nm. In Example 16, the adhesive property of the coating membrane to the nail chip was evaluated after the irradiation with the ultraviolet light at 9.9 J/cm² by using the LED having the wavelength of 340 nm.

The intensity of the ultraviolet light was measured by using the UV light mater (UV-37SD manufactured by CUSTOM corporation), and adjusted by adjusting the irradiation time. In Example 14, Example 15, and Example 16, generation of bubbles was confirmed in the coating membrane after the irradiation of the ultraviolet light. A case where the coating membrane on the nail chip was not peeled off even if the coating membrane was rubbed after the irradiation with the ultraviolet light, and the polishing and the use of the acetone were required for the peeling was indicated as x, and the case where the coating membrane was peeled off when a scratch was made from the end of the nail chip was indicated as ◯. Results are shown in Table 2 below.

TABLE 2

| | Before UV irradiation | After UV irradiation |
|---|---|---|
| Example 14 | ◯ | ◯ |
| Example 15 | ◯ | ◯ |
| Example 16 | ◯ | ◯ |
| Comparative Example 4 | ◯ | X |
| Comparative Example 5 | X | ◯ |

From Table 2, in Example 14, Example 15, and Example 16, the coating membrane firmly adhered to the nail chip before the irradiation with the ultraviolet light, and the coating membrane could be easily peeled off after the irradiation with the ultraviolet light. From the result, it can be seen that the solidified matters of the resin compositions for cosmetics of Example 12 and Example 13 firmly adhered to the nail tip before application of the ultraviolet light as the external stimulus, and adhesive force to the nail tip can be reduced by applying the ultraviolet light. On the other hand, the gel nail of Comparative Example 4 including no stimulus-responsive material firmly adhered to the nail tip, and the strength of adhesion could not be reduced by the irradiation with the ultraviolet light. Furthermore, the coating membrane of the gluing agent of Comparative Example 5 could not firmly adhere to the nail chip, and was easily peeled off from the nail chip even before the irradiation with the ultraviolet light.

[Evaluation of Adhesive Property Before and After Heating]

### Example 17

The base coat gel nail having the commercially available urethane acrylate polymer as the main material was used instead of the acrylic polymer, and 4,4′ oxybis(benzenesulfonylhydrazide) as a hydrazine derivative was added, the solid content concentration being 50.0 mass %. Note that the 4,4′ oxybis(benzenesulfonylhydrazide) was used as the stimulus-responsive material. Otherwise, the resin composition for a cosmetic was obtained in a similar manner as in Example 1. A density of 4,4′ oxybis(benzenesulfonylhydrazide) is 1.5 g/cm³. Since the density of the urethane acrylate polymer is generally about 1.1 g/cm³, a volume ratio of 4,4′ oxybis(benzenesulfonylhydrazide) is 42.3 vol %.

The resin composition for a cosmetic of Example 17 is used to be applied onto a substrate in the range of φ 15 mm with the brush for a nail, and the resin composition for a cosmetic was disposed on the substrate. By using the gel nail curing light to irradiate the resin composition for a cosmetic on the substrate with the LED having the wavelength of 390 nm to 400 nm for 2 minutes, the resin composition for a cosmetic on the substrate was cured. By wiping off the resin uncured on the surface with the ethanol, the coating membrane as the solidified matter of the resin composition for a cosmetic was disposed on a substrate.

Furthermore, the water contact angle with respect to the surface of the solidified matter of the resin composition for a cosmetic of the present example was measured by a method similar to the method in Example 7. The water contact angle was 71.1°.

### Example 18

A gel nail obtained by mixing 57.5 mass % of the urethane methacrylate polymer, 40 mass % of ethyl methacrylate, and 2.5 mass % of the diphenyl(2,4,6-trimethylbenzoyl)phosphine oxide was used instead of the base coat gel nail having the commercially available urethane acrylate polymer as the mail material. As the stimulus-responsive material, azodicarbonamide was used as the azo compound was used instead of the 4,4′ oxybis(benzenesulfonylhydrazide) as the hydrazine derivative. The azodicarbonamide was added to the above gel nail to be 25 mass %. Otherwise, the resin composition for a cosmetic of Example 18 was obtained in a similar manner as in Example 17. A density of the azodicarbonamide is 1.65 g/cm³. Since the gel nail prepared has a density of 1.0 g/cm³, a volume ratio of the azodicarbonamide is 17.1 vol %.

Furthermore, the water contact angle with respect to the surface of the solidified matter of the resin composition for a cosmetic of the present example was measured by a method similar to the method in Example 7. The water contact angle was 70.9°.

### Comparative Example 6

The base coat gel nail was used that has the commercially available urethane acrylate as the mail material without addition of the 4,4′ oxybis(benzenesulfonyl hydrazide), and the base coat gel nail was cured in a similar manner as in Example 17 to dispose the gel nail on the substrate.

For the coating membrane as, for example, the solidified matter of the resin composition for a cosmetic disposed on the substrate of Example 17, Example 18, and Comparative Example 6, the adhesive property before and after the heating with the ultraviolet light was evaluated. A case where the coating membrane on the substrate was not peeled off even if the coating membrane was rubbed before the heating, and the polishing and the use of acetone were required for the peeling was indicated as ◯, and a case where the coating membrane was peeled off when a scratch was made from an end of the coating membrane was indicated as x.

In addition, the heating was performed at 150° C. for 5 minutes by using a hot plate. In Example 17 and Example 18, the generation of the bubbles was found in the coating membrane during the heating. A case where the coating membrane on the substrate was not peeled off even if the coating membrane was rubbed after the heating, and the polishing and the use of acetone were required for the peeling was indicated as x, and the case where the coating membrane was peeled off when a scratch was made from the end of the coating membrane was indicated as ◯. Results are shown in Table 3 below.

TABLE 3

| | Before heating | After heating |
|---|---|---|
| Example 17 | ◯ | ◯ |
| Example 18 | ◯ | ◯ |
| Comparative Example 6 | ◯ | X |

From Table 3, in Example 17 and Example 18, the coating membrane firmly adhered to the substrate before the heating, and the coating membrane could be easily peeled off after the heating. From the result, it can be seen that the solidified matters of the resin compositions for cosmetics of Example 17 and Example 18 firmly adhered to the substrate before application of heat as the external stimulus, and adhesive force to the substrate can be reduced by the heating. On the other hand, the gel nail of Comparative Example 6 including no stimulus-responsive material firmly adhered to the substrate, and the strength of adhesion could not be reduced by the heating.

### INDUSTRIAL APPLICABILITY

The resin composition for a cosmetic of the present disclosure can be used for, for example, the base coat of the nail. Furthermore, the resin composition for a cosmetic of the present disclosure can also be used for use of, for example, the adhesive for an eyelash extension and the adhesive for a lash.

### REFERENCE MARKS IN THE DRAWINGS

1 nail
2 base coat
3 gel nail
3a color gel
3b top gel
4 thin membrane layer
4a interface
10 stimulus generator

1. A resin composition for a cosmetic, the resin composition being included in the cosmetic to be adhered to a part of a living body,
the resin composition for a cosmetic comprising
a stimulus-responsive material that undergoes a volume change by external stimuli, the external stimuli excluding both an external stimulus due to contact with a solvent and an external stimulus due to contact with a solution,
wherein
a solidified matter of the resin composition for a cosmetic has a surface with a water contact angle between 30° and 110°, inclusive, and
the solidified matter of the resin composition for a cosmetic undergoes the volume change by application of the external stimuli to reduce strength of adhesion of the part of the living body to the resin composition for a cosmetic.

2. The resin composition for a cosmetic according to claim 1, wherein the external stimuli are at least one selected from a group consisting of light, heat, magnetic force, electricity, and external force.

3. The resin composition for a cosmetic according to claim 2, wherein the external stimuli are at least one selected from a group consisting of light and heat.

4. The resin composition for a cosmetic according to claim 3, wherein the external stimuli are light having a wavelength ranging from 280 nm up to, not including 400 nm.

5. The resin composition for a cosmetic according to claim 4, wherein the solidified matter of the resin composition for a cosmetic has a transmittance of more than or equal to 10% for light having any wavelength ranging from 280 nm up to, not including 400 nm.

6. The resin composition for a cosmetic according to claim 1, wherein the resin composition for a cosmetic includes more than or equal to 0.5 vol % of the stimulus-responsive material.

7. The resin composition for a cosmetic according to claim 6, wherein the resin composition for a cosmetic includes more than or equal to 0.9 vol % of the stimulus-responsive material.

8. The resin composition for a cosmetic according to claim 1, wherein the solidified matter of the resin composition for a cosmetic undergoes the volume change in more than or equal to 0.5% of an area of an adhesion portion between the part of the living body and the resin composition for a cosmetic by the application of the external stimuli.

9. The resin composition for a cosmetic according to claim 8, wherein the solidified matter of the resin composition for a cosmetic undergoes the volume change in more than or equal to 0.9% of the area of the adhesion portion between the part of the living body and the resin composition for a cosmetic by the application of the external stimuli.

10. The resin composition for a cosmetic according to claim 1, wherein strength of adhesion of the solidified matter of the resin composition for a cosmetic to the part of the living body is reduced to less than or equal to 50% with respect to strength of adhesion before the application of the external stimuli by the application of the external stimuli.

11. The resin composition for a cosmetic according to claim 1, wherein the solidified matter of the resin composition for a cosmetic undergoes the volume change due to generation of gas in the adhesion portion between the part of the living body and the resin composition for a cosmetic by the application of the external stimuli.

12. The resin composition for a cosmetic according to claim 1, wherein the stimulus-responsive material includes an azide compound.

13. The resin composition for a cosmetic according to claim 12, wherein the stimulus-responsive material includes a glycidyl azide polymer.

14. The resin composition for a cosmetic according to claim 13, wherein the resin composition for a cosmetic includes the glycidyl azide polymer between 0.5 mass % and 50 mass %, inclusive.

15. The resin composition for a cosmetic according to claim 1, wherein the part of the living body is at least one selected from a group consisting of a nail and a lash.

16. A resin composition for a nail comprising the resin composition for a cosmetic according to claim 1.

17. A base coat agent for a gel nail comprising the resin composition for a cosmetic according to claim 1.

18. An adhesive for an eyelash extension comprising the resin composition for a cosmetic according to claim 1.
